(19) United States
(12) Patent Application Publication
(10) Pub. No.: US 2021/0085610 A1
(43) Pub. Date: Mar. 25, 2021

(54) STABLE HOT-MELT EXTRUDATE CONTAINING VALSARTAN AND SACUBITRIL

(71) Applicant: ALFRED E. TIEFENBACHER (GMBH & CO. KG), Hamburg (DE)

(72) Inventors: Bala Ramesha Chary RALLABANDI, Hyderabad (Telangana) (IN); Vamshi Ramana PRATHAP, Mallial (IN); Rajesh Krishna Mohan GOLLAPUDI, Peddapurum Mandal (IN); Hendrik SCHLEHAHN, Hamburg (DE); Dieter RUCHATZ, Hamburg (DE)

(73) Assignee: ALFRED E. TIEFENBACHER (GMBH & CO. KG), Hamburg (DE)

(21) Appl. No.: 16/498,909

(22) PCT Filed: Mar. 29, 2018
(86) PCT No.: PCT/EP2018/058207

§ 371 Date: Sep. 27, 2019

Related U.S. Application Data
(60) Provisional application No. 62/513,605, filed on Jun. 1, 2017.

(30) Foreign Application Priority Data
Mar. 31, 2017 (IN) 201711011677

(51) Int. Cl. A61K 9/20; A61K 31/41; A61K 31/216; A61K 9/10

(52) U.S. Cl. CPC A61K 9/2027; A61K 31/41; A61K 9/2031; A61K 9/10; A61K 9/2054; A61K 31/216

(57) ABSTRACT

The present invention relates to a solid unit dosage form for oral administration (tablet or granules) containing a solid dispersion of valsartan and sacubitril in a polymeric matrix. The solid dispersion is prepared by hot-melt extrusion and may contain the active ingredients preferably in a non-crystalline state. LCZ696, (pseudo)polymorphic forms thereof as well as the individual drugs, e.g. valsartan disodium and sacubitril monosodium, may be subjected to the hot-melt extrusion process.

The present invention relates to a solid dispersion containing valsartan and sacubitril as well as to a pharmaceutical composition containing such a solid dispersion.

The combination valsartan and sacubitril is marketed under the tradename Entresto® in the form of film-coated tablets for the prevention of heart failure in patients with chronic heart failure. Entresto contains the drug combination in the form of a cocrystal consisting of valsartan disodium, sacubitril monosodium and 2.5 molecules water. The cocrystal has been designated as LCZ696; its preparation and physical/chemical properties are described in WO 2007/056546 and in Tetrahedron Letters 2012, 53, 275-276. In the Tetrahedron Letters, it is further reported that a desolvated crystalline form exists because the crystalline structure of LCZ696 is maintained up to the melting temperature (around 138° C.), in spite of the fact that two water molecules are lost during the heating.

Various polymorphic forms, pseudopolymorphic forms of LCZ696, i.e. crystalline forms in which the cocrystal contains either more molecules or less molecules of water than 2.5 molecules, and amorphous forms of LCZ696 are known, which are described in WO 2016/037552, WO 2016/049663, WO 2016/051393, WO 2016/125123, WO 2016/151525, WO 2016/201238, WO 2017/009784 and WO 2017/012917.

The Entresto film-coated tablet is an immediate-release tablet that contains, besides LCZ696, microcrystalline cellulose, low-substituted hydroxypropyl cellulose, crospovidone, magnesium stearate, talc and colloidal silicon dioxide as pharmaceutical excipients. Three strengths of the tablet are marketed, which contain, on the basis of the free acid weight of the drugs, 24 mg/26 mg, 49 mg/51 mg and 97 mg/103 mg of sacubitril/valsartan. WO 2009/061713 discloses an immediate-release tablet containing LCZ696 prepared by direct compression or dry-granulation. In the preparation of the tablet, moisture, excessive heat and high shear forces should be avoided in order to prevent amorphization as well as dissociation of the drug components of LCZ696.

WO 2017/000864 describes a direct compression method for preparing a tablet containing LCZ696, in which a mixture of the drug, a hydrophilic diluent, a binder and a disintegrant is subjected to compression.

WO 2017/012600 discloses a tablet containing a physical mixture of sacubitril or a pharmaceutically acceptable salt thereof and valsartan or a pharmaceutically acceptable salt thereof that can be prepared by direct compression, dry-granulation or wet-granulation. The tablets are very sensitive to moisture, so that packaging under nitrogen atmosphere is recommended in order to prevent the degradation of the drugs.

WO 2017/037596 discloses an amorphous solid dispersion of LCZ696 prepared by rotational distillation, spray-drying or freeze-drying a solution containing LCZ696 and a pharmaceutical excipient such as a polymer or magnesium aluminometasilicate (e.g. Neusilin®).

The objective underlying the present invention was the provision of a solid unit dosage form for oral administration that contains valsartan or a pharmaceutically acceptable salt thereof and sacubitril or a pharmaceutically acceptable salt thereof, optionally in form of a complex of the two active ingredients, in a physically and chemically stable form. It was a further objective of the present invention to provide a physico-mechanical stable solid unit dosage form containing these active ingredients. These objectives are attained by the subject matter as defined in the claims.

The solid unit dosage form of the present invention is an immediate-release solid unit dosage form for oral administration, preferably an optionally film-coated tablet or granules filled in a pouch. The solid unit dosage form contains a solid dispersion comprising valsartan or a pharmaceutically acceptable salt thereof and sacubitril or a pharmaceutically acceptable salt thereof as active ingredients. The active ingredients are dispersed in a matrix containing a polymer, wherein the solid dispersion is prepared by hot-melt extrusion. The expression “solid dispersion” as used herein relates to a drug molecularly dissolved in the solid excipient(s) matrix (solid solution) or a drug dispersed as crystalline or amorphous particles in the solid excipient(s) matrix.

The active ingredients are preferably in a non-crystalline state (i.e. molecularly dissolved or in the faun of amorphous particles). The absence of crystalline drug in the solid dispersion can be determined by differential scanning calorimetry (DSC), powder X-ray diffraction (PXRD) and scanning electron microscopy (SEM), respectively. The solid dispersion preferably comprises the active ingredients in a ratio (mol/mol) of 1:1.

According to a preferred embodiment of the present invention, the solid dispersion contains valsartan disodium and sacubitril monosodium, preferably in a ratio (mol/mol) of 1:1. The solid dispersion is prepared by subjecting a mixture containing the polymer and the active ingredients to hot-melt extrusion, wherein the active ingredients are selected from valsartan disodium, sacubitril monosodium and a complex of valsartan disodium and sacubitril monosodium. U.S. Pat. No. 5,217,996 discloses a process for the preparation of sacubitril and its pharmaceutically acceptable salts, in particular, the monosodium salt of sacubitril, and various salts of valsartan, e.g. its disodium salt, are disclosed in WO 02/06253.

For the preparation of the solid dispersion of the present invention, the complex of valsartan disodium and sacubitril monosodium, either in crystalline or amorphous form, can be used in the hot-melt extrusion process. Alternatively, the individual drugs, i.e. valsartan and sacubitril, or pharmaceutically acceptable salts thereof, either in crystalline or amorphous faun, may be subjected to the hot-melt extrusion process. Examples of a crystalline complex of valsartan disodium and sacubitril monosodium include LCZ696 or a polymorphic or a pseudopolymorphic form thereof, The expression “pseudopolymorphic form” relates to crystalline hydrates of the complex of valsartan disodium and sacubitril monosodium other than the hemipentahydrate LCZ696, which contain either more water molecules or less water molecules than 2.5 molecules in the crystal lattice.

The solid dispersion of the present invention contains a polymer that is preferably selected from polyvinylpyrrolidone, poly(vinylpyrrolidone/vinylacetate), polyvinyl-caprolactam/polyvinylacetate/polyethylene glycol graft copolymer, polyethylene glycol/polyvinyl alcohol graft copolymer, poly(ethylene oxide), poly(ethylene oxide/propylene oxide), macrogolglycerol hydroxystearate, hydroxypropyl methylcellulose, hydroxypropyl cellulose, D-α-tocopheryl polyethylene glycol succinate, poly(butyl methacrylate/2-dimethylaminoethyl methacrylate/methyl methacrylate), poly(ethyl acrylate/methyl methacrylate) and poly(ethyl acrylate/methyl methacrylate/trimethylammonioethyl methacrylate chloride). According to a preferred embodiment of the present invention, the polymer is selected from polyvinylcaprolactam/polyvinylacetate/polyethylene glycol graft copolymer, hydroxypropyl methylcellulose and poly(vinylpyrrolidone/vinylacetate) optionally in admixture with polyethylene glycol. Mixtures of polymers may be contained in the solid dispersion of the present invention.

Sacubitril and their pharmaceutically acceptable salts, such as sacubitril monosodium, as well as the amorphous complex of valsartan disodium and sacubitril monosodium are solid but very hygroscopic substances. These substances become deliquescent and sticky when exposed to air humidity. The polymer constituting the matrix of the solid dispersion of the present invention serves the purpose to protect the active ingredients from moisture. Thus, the present invention also relates to the use of polymers for protecting the active ingredients from moisture-induced chemical and physical (polymorph conversion, recrystallization or amorphization) degradation.

It is well known that the handling and the formulation of hygroscopic and deliquescent, sticky active ingredients into solid pharmaceutical formulations is difficult and requires extensive precautions. When water absorption occurs during manufacturing, the consequences include processing problems such as stickiness, clumping, poor release from punches, poor flow characteristics and poor compressibility. Moreover, the physico-mechanical properties and appearance of solid dosage forms comprising a hygroscopic or deliquescent active ingredient are often insufficient, especially after storage. For example, an insufficient tablet hardness as well as unacceptable crumbling or even liquifying of the solid dosage forms may occur. Thus, the present invention also relates to the use of polymers for reducing or eliminating manufacturing and physico-mechanical stability problems of solid dosage forms, which are associated with the hygroscopicity and deliquescence of sacubitril or salts thereof, such as sacubitril monosodium, or a complex of valsartan disodium and sacubitril monosodium as active ingredients.

The solid dispersion may additionally contain a monomeric plasticizer, e.g. triethylcitrate, triacetin, dibutyl sebacate, diethyl phthalate, glycerylmonostearate, glycerine or propylene glycol, or a polymeric plasticizer such as polyethylene glycol or poly(ethylene oxide/propylene oxide). In order to increase the hygroscopic stability of the solid dispersion, a porous, preferably mesoporous inorganic stabilizer can be incorporated, such as mesoporous silica. A mesoporous material is a material containing pores with diameters between 2 and 50 nm. Suitable mesoporous silica products are commercially available under the tradename Syloid®. As an alternative to mesoporous silica, mesoporous magnesium aluminometasilicate may be used, e.g. the magnesium aluminometasilicates marketed under the tradename Neusilin®. A further alternative is mesoporous magnesium carbonate, which is available under the tradename Upsalite®.

Typically, the solid dispersion contains the active ingredients and the polymer in a weight ratio of 3.5:1 to 1:3.5, preferably from 2:1 to 1:1.5 (total weight of the active ingredients, calculated on the basis of the free acid weight of the drugs, to the total weight of the polymer(s)).

The solid dispersion of the present invention is contained in the solid unit dosage form for oral administration together with a pharmaceutical excipient. Preferably, the pharmaceutical excipient is selected from diluents, disintegrants, mesoporous inorganic hygroscopic-stability increasing substances, lubricants and glidants. The solid unit dosage form may be an optionally film-coated tablet. The film coating may be a moisture-barrier film coating in order to increase the hygroscopic stability of the tablet. Alternatively, the milled extrudate may be filled in sachets without additional pharmaceutical excipients.

Examples of diluents include microcrystalline cellulose, calcium hydrogen phosphate, lactose (anhydrous or monohydrate) and calcium carbonate. Examples of disintegrants include croscarmellose sodium, sodium starch glycolate, polyvinyl-polypyrrolidone (crospovidone) and low-substituted hydroxypropyl cellulose (L-HPC). As glidants silicon dioxide, talc and the like may be used, while magnesium stearate, calcium stearate, stearic acid, sodium stearyl fumarate and glycerol dibehenate are examples of suitable lubricants. Examples of mesoporous inorganic hygroscopic-stability increasing substances include silica products (e.g. Syloid®), magnesium aluminometasilicate products (e.g., Neusilin®) and magnesium carbonate products (Upsalite®).

The solid unit dosage forms of the present invention are contained in blister packages, bottles or sachets made for example from PVC, PVDC, PCTFE, COC, PET, PA, Alu, PE or PP and combinations or multilayer films thereof. These packages may comprise a moisture barrier layer and/or they may be packed together with desiccants.

The present invention further relates to a process for preparing an optionally film-coated tablet. The process comprises the steps:
i) subjecting
   (a) a mixture containing valsartan or a pharmaceutically acceptable salt thereof, sacubitril or a pharmaceutically acceptable salt thereof, and a polymer to hot-melt extrusion, or
   (b) a mixture containing a complex of valsartan disodium and sacubitril monosodium, and a polymer to hot-melt extrusion, or
   (c) a first mixture containing valsartan or a pharmaceutically acceptable salt thereof and a polymer to hot-melt extrusion to obtain a first extrudate, a second mixture containing sacubitril or a pharmaceutically acceptable salt thereof and a polymer to hot-melt extrusion to obtain a second extrudate, optionally subjecting a mixture containing the first extrudate and the second extrudate to hot-melt extrusion,
ii) milling the extrudate obtained in step (i)(a), (b) or (c) to obtain granules,
iii) preparing a mixture containing the granules obtained in step (ii) and a pharmaceutical excipient,
iv) compressing the mixture obtained in step (iii) into the tablet.

Alternatively, the granules obtained in step (ii) may be filled in sachets. Alternatively, the granules obtained in step (ii) may be filled in capsules, wherein the granules obtained in step (ii) may be optionally mixed with a pharmaceutical excipient before filling in capsules.

Typically, the maximum temperature applied in the hot-melt extrusion is 120° C. to 160° C., preferably 130° C. to 150° C. and more preferably 135° C. to 145 ° C. The hot-melt extrusion has to be carried out at a temperature that allows the dissolution of the active ingredients in the polymer-containing matrix. Optionally, a degassing unit may be employed during hot-melt extrusion processing.

The following examples are intended to further illustrate the present invention.

### EXAMPLES

Hot-melt extrusion was performed with a Pharma 11 Twin-screw hot-melt extruder from Thermo Fisher Scientific Inc.

### Examples 1 to 8

| | Ex. 1 | Ex. 2 | Ex. 3 | Ex. 4 | Ex. 5 | Ex. 6 | Ex. 7 | Ex. 8 |
|---|---|---|---|---|---|---|---|---|
| Ingredients | [mg] | [mg] | [mg] | [mg] | [mg] | [mg] | [mg] | [mg] |
| Stage-A (Dry mix | | | | | | | | |
| for extrusion) | | | | | | | | |
| Sacubitril/Valsartan complex | 228.516* | 228.516* | 228.516* | 228.516* | — | — | — | — |
| (crystalline) | | | | | | | | |
| Sacubitril/Valsartan complex | — | — | — | — | 237.553* | — | 237.553* | 237.553* |
| (amorphous) | | | | | | | | |
| Sacubitril monosodium | — | — | — | — | — | 103.664* | — | — |
| Valsartan disodium | — | — | — | — | — | 120.649* | — | — |
| HPMC 15 cps | — | — | — | — | — | — | 142.532 | — |
| Copovidone | 137.110 | 114.258 | 137.110 | 137.110 | 142.532 | 134.588 | — | 142.532 |
| (Kollidon VA 64) | | | | | | | | |
| Polyethylene glycol | — | — | — | 13.711 | — | — | — | — |
| (PEG 3350) | | | | | | | | |
| Colloidal silica (Syloid ® | — | — | — | — | — | — | 5.000 | 5.000 |
| AL-1FP) | | | | | | | | |
| Stage-B (Pre-lubrication/ | | | | | | | | |
| blending) | | | | | | | | |
| Microcrystalline cellulose | 12.374 | 25.226 | 12.374 | 8.663 | 7.915 | 9.099 | 1.915 | 1.915 |
| (Comprecel ® PH 102) | | | | | | | | |
| Low substituted HPC | 10.000 | 10.000 | 10.000 | 10.000 | 10.000 | 10.000 | 10.000 | 10.000 |
| (L-HPC LH11) | | | | | | | | |
| Crospovidone Type A | 20.000 | 20.000 | 20.000 | 20.000 | 20.000 | 20.000 | 20.000 | 20.000 |
| (Polyplasdone ® XL) | | | | | | | | |
| Colloidal anhydrous silica | 4.000 | 4.000 | 4.000 | 4.000 | 4.000 | 4.000 | — | — |
| (Aerosil ® 200) | | | | | | | | |
| Colloidal silica (Syloid ® | — | — | — | — | — | — | 9.000 | 9.000 |
| 244FP) | | | | | | | | |
| Stage-B (Lubrication) | | | | | | | | |
| Talc | 4.000 | 4.000 | 4.000 | 4.000 | 4.000 | 4.000 | 4.000 | 4.000 |
| Magnesium stearate | 4.000 | 4.000 | 4.000 | 4.000 | 4.000 | 4.000 | 4.000 | 4.000 |
| Core Tablet Weight | 420.000 | 410.000 | 420.000 | 430.000 | 430.000 | 410.000 | 434.000 | 434.000 |
| Stage-C (Film-Coating) | | | | | | | | |
| Opadry ® 00F540020 Pink | 16.000 | 16.000 | 16.000 | 16.000 | 16.000 | 16.000 | 16.000 | 16.000 |
| Water, Purified | q.s. | q.s. | q.s. | q.s. | q.s. | q.s. | q.s. | q.s. |
| Film-Coated Tablet Weight | 436.000 | 426.000 | 436.000 | 446.000 | 416.000 | 426.000 | 450.000 | 450.000 |
| Zone [° C.] | | | | | | | | |
| Z2 | 40 | | 40 | | | | | |
| Z3 | 70 | | 70 | | | | | |
| Z4 | 100 | | 100 | | | | | |
| Z5 | 120 | | 120 | | | | | |
| Z6 | 150 | | 140 | | | | | |
| Z7 | 140 | | 130 | | | | | |
| Z8 | 130 | | 120 | | | | | |
| Die [° C.] | 120 | | 120 | | | | | |
| Feed rate | Manual | | | | | | | |
| Screw [rpm] | 150 | 150 | 150 | 150 | 150 | 150 | 150 | 150 |
| Die pressure [?] | 6 | 7 | 4 | 3 | 21 | 16 | 12 | 16 |
| Melt temp. [° C.] | 125 | 124 | 124 | 123 | 122 | 122 | 121 | 123 |
| Torque [%] | 36 | 39 | 35 | 29 | 43 | 51 | 52 | 75 |

*Contain 97 mg sacubitril and 103 mg valsartan, respectively

### Process

1. Stage A materials were sifted through #35 mesh, added into double cone blender and blended for 5 minutes and subsequently processed in HME.

2. The extrudes were milled through 1575 μ, 1143 μ and finally 610 μ-screens.

3. Crospovidone, L-HPC, microcrystalline cellulose and colloidal anhydrous silica were sifted through #35 mesh and blended with milled extrudes in double cone blender for 10 minutes.

4. Magnesium stearate and talc were sifted through 35# mesh and blended with step 3 material in double cone blender for 5 minutes.

5. Step 4 lubricated blend was compressed into tablets on rotary tablet compression machine using suitable tooling and parameters.

6. Core tablets were coated in coating pan using Opadry 00F540020 Pink suspension using Water as solvent.

The extrudes showed a good grindability. The milled extrudes contained the active ingredients in non-crystalline state. The physical and chemical stability was high. The milled extrudes showed good compressibility. No crystalline drugs could be detected in the tablets after storage for two months at 40° C./75% RH in a clear PVC-PVDC packaging, and the chemical stability of the drugs was high. The physico-mechanical stability of the tablets before and after storage for two months at 40° C./75% RH in a clear PVC-PVDC packaging was high.

1. A solid dispersion comprising valsartan or a pharmaceutically acceptable salt thereof and sacubitril or a pharmaceutically acceptable salt thereof as active ingredients, wherein the active ingredients are dispersed in a matrix containing a polymer, and wherein the solid dispersion is prepared by hot-melt extrusion.

2. The solid dispersion according to claim 1, wherein the active ingredients are in a non-crystalline state.

3. The solid dispersion according to claim 1, wherein the solid dispersion comprises the active ingredients in a ratio (mol/mol) of 1:1.

4. The solid dispersion according to according to claim 1, wherein the active ingredients are valsartan disodium and sacubitril monosodium.

5. The solid dispersion according to claim 4, wherein the solid dispersion is prepared by subjecting a mixture containing the polymer and the active ingredients to hot-melt extrusion, wherein the active ingredients are selected from valsartan disodium, sacubitril monosodium and a complex of valsartan disodium and sacubitril monosodium.

6. The solid dispersion according to claim 5, wherein the complex of valsartan disodium and sacubitril monosodium is crystalline or amorphous.

7. The solid dispersion according to claim 6, wherein the crystalline complex of valsartan disodium and sacubitril monosodium is LCZ696 or a polymorphic or a pseudopolymorphic form thereof.

8. The solid dispersion according to claim 1, wherein the polymer is selected from polyvinylpyrrolidone, poly(vinyl-pyrrolidone/vinylacetate), polyvinylcaprolactam/polyvinylacetate/polyethylene glycol graft copolymer, polyethylene glycol/polyvinyl alcohol graft copolymer, poly(ethylene oxide), poly(ethylene oxide/propylene oxide), macrogolglycerol hydroxystearate, hydroxypropyl methylcellulose, hydroxypropyl cellulose, D-α-tocopheryl polyethylene glycol succinate, poly(butyl methacrylate/2-dimethylaminoethyl methacrylate/methyl methacrylate), poly(ethyl acrylate/methyl methacrylate) and poly(ethyl acrylate/methyl methacrylate/trimethylammonioethyl methacrylate chloride).

9. The solid dispersion according to claim 8, wherein the polymer is polyvinylcaprolactam/polyvinylacetate/polyethylene glycol graft copolymer, hydroxypropyl methylcellulose or poly(vinylpyrrolidone/vinylacetate) optionally in admixture with polyethylene glycol, preferably poly(vinylpyrrolidone/vinylacetate) or hydroxypropyl methylcellulose.

10. A solid unit dosage form for oral administration comprising the solid dispersion according to claim 1 and a pharmaceutical excipient.

11. The solid unit dosage form according to claim 10, wherein the pharmaceutical excipient is selected from diluents, disintegrants, lubricants, glidants and mesoporous inorganic hygroscopic-stability increasing substances.

12. The solid unit dosage form according to claim 10, wherein the solid unit dosage form is an optionally film-coated tablet.

13. A process for preparing an optionally film-coated tablet according to claim 11, comprising the steps:
i) subjecting
(a) a mixture containing valsartan or a pharmaceutically acceptable salt thereof, sacubitril or a pharmaceutically acceptable salt thereof, and a polymer to hot-melt extrusion, or
(b) a mixture containing a complex of valsartan disodium and sacubitril monosodium, and a polymer to hot-melt extrusion, or
(c) a first mixture containing valsartan or a pharmaceutically acceptable salt thereof and a polymer to hot-melt extrusion to obtain a first extrudate, a second mixture containing sacubitril or a pharmaceutically acceptable salt thereof and a polymer to hot-melt extrusion to obtain a second extrudate, optionally subjecting a mixture containing the first extrudate and the second extrudate to hot-melt extrusion,
ii) milling the extrudate obtained in step (i)(a), (b) or (c) to obtain granules,
iii) preparing a mixture containing the granules obtained in step (ii) and a pharmaceutical excipient,
iv) compressing the mixture obtained in step (iii) into the tablet.

14. The solid dispersion according to claim 2, wherein the solid dispersion comprises the active ingredients in a ratio (mol/mol) of 1:1.

15. The solid dispersion according to according to claim 2, wherein the active ingredients are valsartan disodium and sacubitril monosodium.

16. The solid dispersion according to according to claim 3, wherein the active ingredients are valsartan disodium and sacubitril monosodium.

17. The solid dispersion according to claim 2, wherein the polymer is selected from polyvinylpyrrolidone, poly(vinylpyrrolidone/vinyl acetate), polyvinylcaprolactam/polyvinylacetate/polyethylene glycol graft copolymer, polyethylene glycol/polyvinyl alcohol graft copolymer, poly(ethylene oxide), poly(ethylene oxide/propylene oxide), macrogolglycerol hydroxystearate, hydroxypropyl methylcellulose, hydroxypropyl cellulose, D-α-tocopheryl polyethylene glycol succinate, poly(butyl methacrylate/2-dimethyl-aminoethyl methacrylate/methyl methacrylate), poly(ethyl acrylate/methyl methacrylate) and poly(ethyl acrylate/methyl methacrylate/trimethylammonioethyl methacrylate chloride).

18. The solid dispersion according to claim 3, wherein the polymer is selected from polyvinylpyrrolidone, poly(vinylpyrrolidone/vinyl acetate), polyvinylcaprolactam/polyvinylacetate/polyethylene glycol graft copolymer, polyethylene glycol/polyvinyl alcohol graft copolymer, poly(ethylene oxide), poly(ethylene oxide/propylene oxide), macrogolglycerol hydroxystearate, hydroxypropyl methylcellulose, hydroxypropyl cellulose, D-α-tocopheryl polyethylene glycol succinate, poly(butyl methacrylate/2-dimethyl-aminoethyl methacrylate/methyl methacrylate), poly(ethyl acrylate/methyl methacrylate) and poly(ethyl acrylate/methyl methacrylate/trim ethylammonioethyl methacrylate chloride).

19. The solid dispersion according to claim 4, wherein the polymer is selected from polyvinylpyrrolidone, poly(vinylpyrrolidone/vinyl acetate), polyvinylcaprolactam/polyvinylacetate/polyethylene glycol graft copolymer, polyethylene glycol/polyvinyl alcohol graft copolymer, poly(ethylene oxide), poly(ethylene oxide/propylene oxide), macrogolglycerol hydroxystearate, hydroxypropyl methylcellulose, hydroxypropyl cellulose, D-α-tocopheryl polyethylene glycol succinate, poly(butyl methacrylate/2-dimethyl-aminoethyl methacrylate/methyl methacrylate), poly(ethyl acrylate/methyl methacrylate) and poly(ethyl acrylate/methyl methacrylate/trim ethylammonioethyl methacrylate chloride).

20. The solid dispersion according to claim 5, wherein the polymer is selected from polyvinylpyrrolidone, poly(vinylpyrrolidone/vinyl acetate), polyvinylcaprolactam/polyvinylacetate/polyethylene glycol graft copolymer, polyethylene glycol/polyvinyl alcohol graft copolymer, poly(ethylene oxide), poly(ethylene oxide/propylene oxide), macrogolglycerol hydroxystearate, hydroxypropyl methylcellulose, hydroxypropyl cellulose, D-α-tocopheryl polyethylene glycol succinate, poly(butyl methacrylate/2-dimethyl-aminoethyl methacrylate/methyl methacrylate), poly(ethyl acrylate/methyl methacrylate) and poly(ethyl acrylate/methyl methacrylate/trim ethylammonioethyl methacrylate chloride).
